(19) United States
(12) Patent Application Publication
(10) Pub. No.: US 2014/0230849 A1
(43) Pub. Date: Aug. 21, 2014

(54) Process for cleaning and sanitizing surgical instruments in general and device suited to implement said process

(71) Applicant: Alessandro Paolo Sommacal, Padova (IT)

(72) Inventor: Alessandro Paolo Sommacal, Padova (IT)

(73) Assignee: BICAR JET S.R.L., Padova (IT)

(21) Appl. No.: 14/342,353

(22) PCT Filed: Mar. 29, 2012
(86) PCT No.: PCT/IB2012/051514

§ 371 Date: Feb. 28, 2014

(30) Foreign Application Priority Data
Nov. 11, 2011 (IT) PD2011A000352

(51) Int. Cl. A61B 19/00

(52) U.S. Cl. CPC A61B 19/34; A61L 2202/24
USPC 134/7; 15/302

(57) ABSTRACT

The invention concerns a process for cleaning and sanitizing surgical tools or surgical instruments in general, and a device suited to implement the process. In particular, the invention includes the use of an abrasive cleaning material, emitted under pressure and at high speed against the instruments for abrading and removing the substances that adhere to the surfaces of the same instruments, wherein the abrasive cleaning material includes sodium bicarbonate salts, mixtures of the same or similar substances.

### FIELD OF THE INVENTION

The present patent concerns the processes and devices for cleaning surgical tools or surgical instruments, and in particular it concerns a new sand blasting process for cleaning and sanitizing surgical tools or surgical instruments in general, and a device suited to implement said process.

### BACKGROUND OF THE INVENTION

Surgical tools and instruments used in the medical field, for example in surgery, dentistry, veterinary medicine, etc, are known, which are used for carrying out operations on the patient's body.

In order to be used, said instruments must be completely and accurately cleaned and sanitized, meaning that their surface must not present any type of foreign substance, bacteria or other elements that may cause pathological reactions in any type of patient.

Therefore, surgical tools must be cleaned again and sanitized accurately after each use, as well as packaged individually in sterile and sealed packages suited to be quickly opened to extract the tools at the moment of use by the surgeon or operator in general.

In particular, before undergoing the sanitization process, which usually is performed with high temperature steam or other chemical or thermal systems, the surgical instruments must be previously cleaned very accurately in order to completely remove any organic residues from their metal surface.

It is in the organic residues, in fact, if they are not removed before sanitization, that the molecule breaking process takes place during the heat treatment, with the formation of substances that affect the cleanliness and sterility of the surgical instrument and considerably increase corrosion problems.

In order to protect the health of medical personnel against possible infections due to contact, even if accidental, with infected surgical instruments, automatic instrument washing devices have been designed that are used for cleaning and sanitizing the instruments.

These devices usually operate in three steps. During the first step the surgical instruments are washed with suitable detergents and rinsed with water, which must ensure complete removal of the organic residues present on the instruments.

A drawback posed by the known processes using chemical products lies in that some of them, commonly used in washing cycles, are too aggressive against the stainless steel used for making surgical instruments and cause these to corrode.

Furthermore, the surgical instruments comprise portions of their surface that are smooth and other portions that are rough, for example at the level of the handpieces, where better hold must be guaranteed to the operator. Some surgical instruments also comprise interstices and small spaces according to the specific shape of the instrument and to usage needs.

Organic impurities accumulate in said interstices and on said rough surface and are difficult to remove with the known washing processes.

Due to the fact that the cleansing systems used are not perfectly effective, before the washing operations a manual mechanical operation is often necessary and this must be performed by a specialized operator. This operation consists in the manual brushing of the instruments, which subjects the operator to the risk of infections, due to possible cuts, abrasions, punctures or any other accidental event deriving from the brushing operation. Furthermore, the brushing operation does not always guarantee the complete removal of organic residues, since the tools used for this mechanical operation cannot always reach the contaminated interstices or cavities and/or sometimes the encrustation resulting from the drying of the organic substances are especially adherent to the surface.

For this reason systems are also known that use softening baths suited to favor the mechanical and/or chemical removal of organic residues from the most complex surfaces, however these methods are not always effective. The contaminations left, especially in the interstices and cavities, are then fixed with the sanitizing heat treatment and therefore the effectiveness of this last step, so important for the health of the patients that will be treated subsequently, cannot be guaranteed.

The second, neutralizing step, includes the use of acid products that serve to solubilize and completely remove the oxides formed during cleaning with detergents.

The acid solutions used, however, have also a corrosive effect, in particular at the level of the contact points between metal and metal or between metal and plastic, where the conditions may facilitate the occurrence of localized corrosion phenomena.

The third step includes the cleansing of the instruments with high temperature water, approximately at 90°, which may cause more marked corrosion effects at the level of said areas subjected to localized corrosion.

Therefore, the known processes and equipment for cleaning and sanitizing surgical instruments pose several drawbacks.

### SUMMARY OF THE INVENTION

In order to overcome said drawbacks, a new type of sand blasting process has been studied and implemented, which uses sodium bicarbonate for cleaning and sanitizing surgical tools or surgical instruments in general, in addition to a device for implementing said process.

The main object of the present invention is to ensure highly effective mechanical removal of the substances stuck to the entire surface of the instruments and, at the same time, a highly effective washing, sanitizing and disinfecting process, if any.

It is a further object of the present invention to guarantee a long-lasting disinfecting and anti-bacterial action, intended to hinder a new proliferation of bacteria on the surfaces of the instruments.

A further important advantage of the present invention also consists in the considerable reduction of washing times and thus of the related costs, as the new product performs the abrading step, the cleansing step and if necessary also the disinfecting and/or biocidal action at the same time, with a single application.

Another advantage deriving from the use of the present invention lies in the reduction of energy costs, as the effectiveness of the product is guaranteed even if the product is applied with air, with or without water, not heated, and thus with no need to use hot water.

These and other direct and complementary objects are achieved by the new sand blasting process which uses sodium bicarbonate for cleaning and sanitizing surgical tools or surgical instruments in general, and by a device for implementing said process.

According to the new cleaning process, the removal of the substances adhering and stuck to the surfaces of the instrument to be cleaned takes place through abrasion, with the ejection of abrasive cleaning material at high speed against the instruments to be cleaned.

In particular, said abrasive cleaning material comprises sodium bicarbonate salts, mixtures of the same or the like.

Said sodium bicarbonate salts can be used both dry and wet, in the latter case dissolved in water beyond the saturation limit, so as to form a solution comprising dissolved and undissolved salts, suited to be emitted under pressure and at high speed and ejected against the instruments to be cleaned. The portion of sodium bicarbonate salts dissolved in the solution performs the real washing function, dissolving and leaching the organic material present on the surface of the instruments.

The portion of undissolved sodium bicarbonate salts, instead, serves as abrasive material.

The new process is particularly suitable for washing said surgical instruments, as it does not involve any corrosive effect on the material from which said instruments are made and there is no abrasive action, meaning no surface modification of the instrument.

Furthermore, sodium bicarbonate has a strong sanitizing, antibacterial and antifungal action.

Furthermore, sodium bicarbonate is not dangerous for operators and is not detrimental to the environment, is 100% biodegradable and soluble, which means that it is not polluting and not subjected to special restrictions for storage, transport and handling.

According to a possible embodiment of the invention, in the new process said abrasive cleaning material comprises, in addition to sodium bicarbonate salts and water, even one or more further disinfecting and/or biocidal agents, for example in a concentration included between 0.1 and 25%.

In this way, with a single step it is possible to carry out both the instrument washing operation and the disinfecting operation.

According to the invention, said abrasive cleaning material may also comprise one or more further substances with cleansing and/or disinfecting properties and/or one or more specifically acting medical products.

The water used may be heated or at room temperature, since the use of sodium bicarbonate, in particular with the addition of disinfecting and/or biocidal agents, guarantees optimal cleaning and sanitizing results.

Therefore, the new process also ensures savings in energy and running costs.

The elimination of the brushing process results in the elimination of the risks for operators deriving from possible infections due to accidental punctures, abrasions or cuts that may occur during the brushing operation, with a clear reduction of any accident related costs.

The improved effectiveness of the cleaning process also generates a considerable reduction in the percentage of infections in patients treated subsequently, thanks to better and more effective cleaning of the surgical instruments, with a consequent reduction of the related costs.

The process may comprise a final step in which the instruments are rinsed with cold water, meaning non heated water, and/or are blown with dry sodium bicarbonate salts.

### BRIEF DESCRIPTION OF THE DRAWINGS

FIG. 1 illustrates a device according to the invention.

### DETAILED DESCRIPTION OF A PREFERRED EMBODIMENT

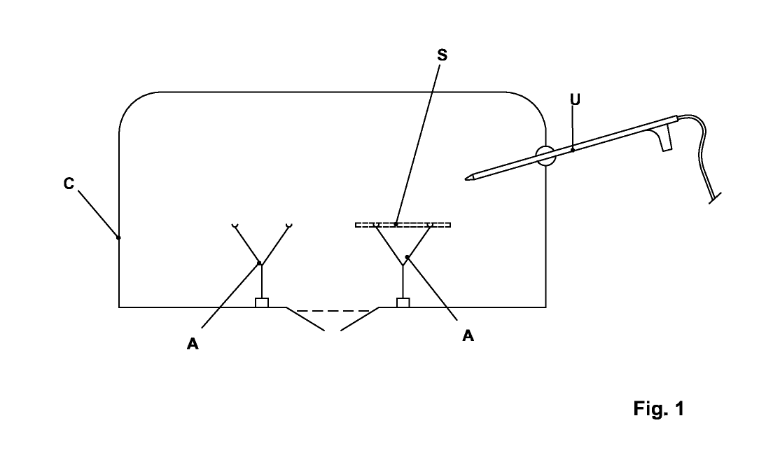

For the implementation of the new process for cleaning and sanitizing surgical instruments a device, shown in FIG. 1, is used that comprises a closed sand blasting cabinet (C) suited to accommodate one or more instruments to be cleaned.

According to the invention, inside said cabinet there is/are one or more supports (A) for said surgical instruments (S), suited to constrain the instruments and keep them in position during the sand blasting operation.

Said supports (A) can be of the fixed or movable type, for example they can be rotated or translated, so as to expose the entire surface of the instrument to the jet emitted by the emitter nozzle (U).

According to a possible embodiment of the invention, said emitter nozzle is movable, that is, travels along the three spatial directions and/or rotates, so as to direct the jet on the entire surface of the instruments to be cleaned. Said instruments to be cleaned can in turn be constrained to said fixed or movable supports, or held manually by the user.

Said emitter nozzle can be moved manually or can be automated.

According to a further alternative embodiment, said emitter nozzle is fixed inside the cabinet, and thus the jet is permanently oriented in the same direction. According to this solution, the instruments to be cleaned can be maneuvered manually by the operator, in order to expose their entire surface to the jet. Alternatively, said supports of the instruments to be cleaned are mechanized and movable, translating and/or rotating, thus completely automating the process.

The device also comprises at least one abrasive material feed system, in turn comprising an abrasive material tank, and wherein, through at least one duct, a flow of pressurized air and/or water draws or thrusts said abrasive material from said tank and transports it to an emitter nozzle located inside said cabinet.

By means of said emitter nozzle said abrasive material is ejected at high speed, inside said sand blasting cabinet, on said instruments to be cleaned. The device may also comprise a vacuum suction system for drawing the abrasive material inside said cabinet and an air filtering system for filtering the air flowing out of the cabinet and recovering the material used in at least one apposite container.

Said sand blasting cabinet can have any shape and size and comprises a casing with at least one access door for introducing and extracting the surgical instruments.

Said casing of the cabinet comprises also one or more check windows, one or two openings with sleeve glove suited to allow the operator to maneuver the sand blasting nozzle and/or to rotate the instruments to be cleaned inside the cabinet, and one discharge opening on the bottom of the cabinet.

Said bottom of the cabinet can be shaped, for example, so as to favor the outflow of the waste material.

The cleaning operations with sodium bicarbonate salts can thus be carried out inside said cabinet, both manually, by one or more operators at the same time, and in an automated manner, with the aid of appropriate instruments or suitable automated mechanical systems.

Therefore, with reference to the above description, the following claims are expressed.

The invention claimed is:

1. A process for cleaning and sanitizing surgical tools or surgical instruments, comprising:
providing an abrasive cleaning material; and
emitting said abrasive cleaning material under pressure and at high speed against said instruments for abrading and removing substances that adhere to surfaces of the instruments,
wherein said abrasive cleaning material comprises sodium bicarbonate salts, or a mixture thereof

2. The process according to claim 1, wherein said abrasive cleaning material is provided in a dry state.

3. The process according to claim 1, wherein said abrasive cleaning material comprises said sodium bicarbonate salts dissolved in water beyond saturation limit so as to form a solution comprising one or both of dissolved or undissolved salts, suited to be emitted under pressure and at high speed and projected against the instruments to be cleaned, and wherein said water is or is not heated.

4. The process according to claim 1, wherein said abrasive cleaning material also comprises one or more additional agents with disinfecting or biocidal properties.

5. The process according to claim 4, wherein said one or more additional agents agent are present in a concentration included between 0.1 and 25%.

6. The process according to claim 5, wherein said abrasive cleaning material also comprises one or more additional substances with cleansing or disinfecting properties or one or more specifically acting medical products.

7. The process according to claim 1, further comprising at least one final step of rinsing the instruments with water or a blowing step for drying.

8. A device for cleaning and sanitizing surgical instruments using the process according to claim 1, comprising:
at least one closed sand blasting cabinet (C), suited to accommodate one or more of said instruments (S) to be cleaned, said blasting cabinet comprising a casing with at least one access door for introducing the instruments (S) to be cleaned; and
at least one system for supplying sodium bicarbonate salts or a mixture thereof, said system comprising a tank containing sodium bicarbonate salts or a mixture thereof, a duct through which pressurized air or water flow draws or pushes the sodium bicarbonate salts or the mixture thereof out of said tank to transport said sodium bicarbonate salts or said mixture thereof to an emitter nozzle (U) positioned inside said sand blasting cabinet (C),
wherein said emitter nozzle (U) ejects said sodium bicarbonate salts or the mixture thereof at high speed inside said sand blasting cabinet (C), onto said instruments (S) to be cleaned.

9. The device for cleaning and sanitizing surgical instruments according to claim 8, wherein said sand blasting cabinet (C) comprises at least one opening with a sleeve glove suited to allow an operator to maneuver said emitter nozzle (U) or the instruments (S) to be cleaned inside the sand blasting cabinet.

10. The device for cleaning and sanitizing surgical instruments according to claim 8, wherein said emitter nozzle (U) is configured to be moved in an automated way, and translated along three spatial directions or rotated, so as to direct a jet on an entire surface of the instruments (S) to be cleaned.

11. The device for cleaning and sanitizing surgical instruments according to claim 8, wherein said emitter nozzle (U) is fixed, maintaining a jet direction unchanged.

12. The device for cleaning and sanitizing surgical instruments according to claim 8, further comprising one or more supports (A) for said surgical instruments (S) inside said sand blasting cabinet (C), configured to constrain the instruments (S) and keep the instruments in the correct position during sand blasting.

13. The device for cleaning and sanitizing surgical instruments according to claim 12, wherein said supports (A) for the instruments (S) to be cleaned are configured to be moved in an automated way, through rotation or translation, so as to expose an entire surface of one of the instruments (S) to a jet of the emitter nozzle (U).

14. The device for cleaning and sanitizing surgical instruments according to claim 8, further comprising:
means for rinsing or blowing on one of the instruments after sand blasting;
a vacuum suction system or a pressure thrust system configured to convey the sodium bicarbonate salts or the mixture thereof into said sand blasting cabinet (C); and
a filtering system for filtering air that flows out of the sand blasting cabinet (C) and recovering used material, for which at least one suitable container is provided.
